(19) United States
(12) Patent Application Publication
(10) Pub. No.: US 2015/0351826 A1
(43) Pub. Date: Dec. 10, 2015

(54) BIPOLAR RESECTOSCOPE

(71) Applicant: BOWA-ELECTRONIC GMBH & CO. KG, Gomaringen (DE)

(72) Inventors: Thomas Kroeber, Gomaringen (DE); Alexander Doppelstein, Bodelshausen (DE); Martin Heinrich, Hechingen (DE)

(21) Appl. No.: 14/762,957

(22) PCT Filed: Jan. 20, 2014
(86) PCT No.: PCT/EP2014/051048

§ 371 Date: Jul. 23, 2015

(30) Foreign Application Priority Data
Jan. 24, 2013 (DE) 10 2013 001 156.6

(51) Int. Cl. A61B 18/14; A61B 18/12; A61B 1/00

(52) U.S. Cl. CPC A61B 18/149; A61B 1/00087; A61B 18/1206; A61B 18/1482; A61B 1/00124; A61B 2018/126; A61B 2018/00083

(57) ABSTRACT

A bipolar resectoscope has an inner shaft with an insulating insert at its distal end and an electrode transporter that can be arranged in the inner shaft. A first electrode is longitudinally moveable in the electrode transporter and has a proximal end that can be connected to a first connection of a high-frequency generator and a second electrode that can be connected at its proximal end to a second connection of the high-frequency generator. A circumferential electrically conductive electrode surface (29) is exposed transverse to the longitudinal axis (28) of the inner shaft (2) and is connected to the distal end (31) of the second electrode (5) on the inner side (30) of the insulating insert (8). The exposed electrode surface (29) of the insulating insert (8) is on the side (34) facing away from the longitudinal axis (33) of the insulating insert, and is insulated electrically toward the inside.

### BACKGROUND

1. Field of the Invention

The invention relates to a bipolar resectoscope, comprising
an inner shaft having at its distal end an insulating insert made of an electrically non-conductive material,
an electrode transporter that can be arranged in the inner shaft,
a first electrode, which can be arranged in a longitudinally movable manner in the electrode transporter and which can be connected, at its proximal end facing away from the distal end, to a first connection of a high-frequency generator, and
a second electrode, which can be connected at its proximal end to a second connection of the high-frequency generator.

2. Description of the Related Art

U.S. Pat. No. 6,827,717 discloses a urological resectoscope with a first electrode that can also be designed as a bipolar electrode. Such resectoscopes consist of an endoscopic shaft that is insertable into the urethra and into which can be placed an electrode transporter with a bipolar electrode and an optical system (endoscope) arranged in a guide tube. The electrode transporter has a sliding body that is movable in a longitudinal direction along the guide tube of the electrode transporter, said sliding body having a receptacle located parallel to the longitudinal axis of the guide tube to accommodate a proximal end of the electrode facing the operator. The proximal end of the electrode is fixable within the sliding body by way of a fixing device. At its distal end, the known resectoscope has an insulating insert made of an electrically non-conductive material.

It is problematic with respect to a bipolar resectoscope that in order to achieve high cutting effectiveness, the active or cutting electrode has a relatively small cutting surface and the passive or neutral electrode must be designed with a relatively large surface in order to prevent it from exerting its own cutting effect.

U.S. Pat. No. 6,471,701 discloses a bipolar resectoscope whose loop-shaped cutting electrode, as the first electrode, has a roof-shaped insulation carrier above its semicircular cutting loop, said insulation carrier having an exposed electrode surface on its side facing away from the cutting loop, and said exposed electrode surface acting as a passive or neutral electrode whose distal end is connected to a second electrode.

It is disadvantageous in this connection that, on the one hand, the neutral electrode must be replaced along with the cutting electrode, and on the other hand, the additional insulation carrier with the exposed electrode surface undesirably restricts the field of view of the endoscopic optical system.

U.S. Pat. No. 7,611,511 discloses a bipolar resectoscope that also has a loop-shaped cutting electrode as the active electrode and a tape-shaped electrode as the passive electrode, the latter being placed in front of the cutting loop in the distal direction and arranged radially above the cutting loop. This known resectoscope also exhibits the disadvantages mentioned above.

Furthermore, U.S. Pat. No. 5,902,272 discloses a bipolar resectoscope that also has an active electrode that is a loop-shaped cutting electrode arranged so as to be movable longitudinally within an inner shaft by means of an electrode transporter. The distal end of an outer shaft that is set back in a proximal direction relative to the inner shaft serves as a passive electrode. The electrically conductive outer shaft is insulated toward the outside, with the exception of its distal end.

It is disadvantageous in this connection, on the one hand, that the spacing in the longitudinal direction between the first and second electrode has to be relatively large and is undesirably displaced in the proximal direction, and on the other hand, that defective insulation of the outer shaft can result in undesired current densities and therefore in undesired burns.

Furthermore, U.S. Pat. No. 4,116,198 discloses a bipolar resectoscope that also has as its active electrode a loop-shaped cutting electrode that is arranged within an endoscope shaft so as to be longitudinally movable by means of an electrode transporter. An electrically conductive piece of tubing arranged between a distal insulating insert and the distal end of an endoscope shaft serves as a passive or neutral electrode.

In this connection, it is disadvantageous that the electrically conductive piece of tubing must be electrically isolated from the distal end of the endoscope shaft by means of an additional insulating ring. As an additional part, this insulating ring must be connected to the distal end of the endoscope shaft, on the one hand, and to the proximal end of the piece of tubing facing toward the distal end of the endoscope shaft, on the other hand, and the connection is relatively costly to make. Moreover, it is also disadvantageous that the electrically conductive piece of tubing conducts electricity both in a radial direction outwards as well as in a radial direction inwards, which in combination with the surrounding tissue and the position of the endoscope shaft, results in conductive surfaces with different conductivities and therefore in different and fluctuating current densities.

The problem which the present invention seeks to solve is, therefore, to further refine a generic bipolar resectoscope such that the second electrode, as the passive electrode, has a sufficiently large electrode surface and is positioned as close as possible to the first electrode, which is designed as an active electrode. The intended result is to achieve electrical safety, on the one hand, as well as the least possible impairment of the field of view, on the other hand, in a cost-effective manner.

### SUMMARY

This problem is solved in conjunction with a bipolar resectoscope with an inner shaft having a distal end with an insulating insert made of an electrically non-conductive material, an electrode transporter that can be arranged in the inner shaft, and first and second electrodes. The first electrode is longitudinally moveable in the electrode transporter. A proximal end of the first electrode can be connected to a first connection of a high-frequency generator. The distal end of the first electrode is bifurcated and has two parallel loop guide tubes parallel to the longitudinal axis and between which a semicircular cutting loop is tensioned. The second electrode has a proximal end that can be connected to a second connection of the high-frequency generator. The insulating insert has at its distal end a circumferential, electrically conductive electrode surface that is exposed transverse to the longitudinal axis of the inner shaft, said exposed electrical surface being connected to the distal end of the second electrode on the inner side of the insulating insert, and in that the exposed electrode surface of the insulating insert is arranged on the outer side, facing radially away from the longitudinal axis, of the insulating insert, and is electrically insulated toward the inside, or in that the exposed electrode surface of the insulating insert is arranged on the inner side, facing radially toward the longitudinal axis, of the insulating insert, and is electrically insulated toward the outside.

The arrangement of an exposed electrode surface on the insulating insert of the inner shaft results in a sufficiently large passive electrode surface proximate to the first electrode without restricting the view, as compared to the use of a conventional insulating insert that is completely non-conductive. This results in very good cutting characteristics in tissue that come as a surprise to a person skilled in the art. In this connection, the outer shaft can be completely conductive or it can also be made completely of a non-conductive material. In both cases, no electricity is conducted by the outer shaft. This also results in a high degree of electrical safety and protection from undesired burns caused by conduction of electricity by the outer shaft. Insulating the exposed electrode surface on the outer side or the inner side of the insulating insert provides a defined electrode surface in each case. It is surprising here that, despite the short electrical pathways between the two electrodes, this does not result in a current flow that would impair cutting performance. No additional, relatively costly, insulating ring vis à vis the inner shaft is required.

According to a preferred embodiment of the invention, the first electrode is designed as an active cutting electrode and the exposed electrode surface of the insulating insert is designed as a passive neutral electrode.

According to another embodiment of the invention, the exposed electrode surface of the insulating insert is arranged in an insulated manner, in the longitudinal direction, on the one hand toward the distal end of the insulating insert, and on the other hand toward the free end of the inner shaft, i.e. toward the proximal end of the insulating insert.

According to another embodiment of the invention, the insulating insert is made of plastic and the exposed electrode surface is made of a metallic material. The electrode surface can then be embedded in the insulating surface.

Alternatively, the insulating insert can be made of a non-conductive ceramic material and the exposed electrode surface can be made of a metalized, and therefore conductive, ceramic material.

According to another preferred embodiment of the invention, the inner shaft can be arranged within an outer shaft and both shafts form a continuous flushing shaft with continuous flushing.

According to another preferred embodiment of the invention, the distal end of the outer shaft is set back, in the proximal direction, relative to the distal end of the insulating insert in the area of the insulating insert. In this connection, the distal end of the outer shaft has a plurality of return flow openings. The return flow openings enable a return flow of flushing fluid between the inner shaft and the outer shaft.

According to another preferred embodiment of the invention, the inner shaft is made of an electrically conductive material. This is also advantageous in combination with an outer shaft made of an electrically conductive material.

According to another preferred embodiment of the invention, the proximal-side end of the insulating insert is inserted into the distal end of the inner shaft, and the adapter connected to the electrode surface is connected to the distal end of the second electrode by a plug connector.

This enables simple replacement of the insulating insert with the electrode surface. Different insulating inserts with different electrode surfaces can also be used.

Further features and advantages of the invention may be derived from the following specific description and from the drawings.

### BRIEF DESCRIPTION OF THE DRAWINGS

FIG. 1 is a lateral view of a bipolar resectoscope with an optical system indicated by a dotted line.

FIG. 2 is an enlarged cross-sectional, lateral view of the distal end of FIG. 1.

FIG. 3 is a lateral view of a second embodiment of a bipolar resectoscope with an optical system indicated by a dotted line.

FIG. 4 is an enlarged cross-sectional, lateral view of the distal end of FIG. 3.

FIG. 5 is a lateral view of a third embodiment of a bipolar resectoscope with an optical system indicated by a dotted line.

FIG. 6 is an enlarged cross-sectional, lateral view of the distal end of FIG. 5.

FIG. 7 is a cross-sectional, lateral view of the bipolar resectoscope from FIG., without an optical system.

FIG. 8 is an enlarged lateral view of the distal end of an inner shaft with insulating insert.

FIG. 9 is a cross-sectional, lateral view of the distal end of FIG. 8.

FIG. 10 is a lateral view of the distal end of FIG. 8 with pulled-out insulating insert.

FIG. 11 is a cross-sectional, lateral view of the distal end of FIG. 10.

### DETAILED DESCRIPTION

A bipolar resectoscope 1 essentially consists of an inner shaft 2, an electrode transporter 3, a first electrode 4, a second electrode 5 and an optical system 6.

At its distal end 7 facing the patient, the inner shaft 2 possesses an insulating insert 8. The electrode transporter 3 is insertable into the inner shaft 2 from the proximal end 9 of the latter, facing away from the distal end 7, and the electrode transporter 3 can be fastened to the inner shaft 2.

The electrode transporter 3 has a guide tube 10 on whose proximal end 11, facing the operator, a connector piece 12 is arranged to connect the optical system 6, which is mounted in the guide tube 10. The electrode transporter 3 has a finger grip 13 and a connection cone 14 placed in front of it in the distal direction, via which cone the electrode transporter 3 can be locked to a main body 15 that forms the proximal end of the inner shaft 2.

A sliding body 16 that can be moved longitudinally is arranged on the guide tube 10. The sliding body 16 is connected to the connection piece 12 by a spring joint 17 and can be pressed in the direction of the finger grip 13 by way of a thumb ring 18 against the spring force of the joint 17. The sliding body 16 has a receptacle for accommodating a proximal end 19 of the first electrode 4. The sliding body 16 has a guide channel 20 by means of [which] the sliding body 16 is guided along the guide tube 10, the longitudinal axis 21 of the guide tube 10 coinciding with the longitudinal axis of the guide channel 20. Transverse to the longitudinal axis 21, the sliding body 16 has a plug socket 22 for a plug (not shown) which can be connected at an instrument-side end of a high-frequency cable to a first connection of a high-frequency generator (also not shown). Correspondingly, the proximal end of the second electrode 5 can be connected to a second connection of the high-frequency generator.

The first electrode 4 is bifurcated in known fashion at its distal end 23 and has, approximately parallel to the longitudinal axis 21, two parallel loop guide tubes 24, between which a semicircular cutting loop 25 is tensioned. In the embodiments, the cutting loop 25 is bent back in a proximal direction and forms an acute angle 26 with respect to the longitudinal axis 21 and/or with respect to the loop guide tubes 24.

The insulating insert 8 has, at its distal end 27, a circumferential electrically conductive electrode surface 29 that is exposed transversely to the longitudinal axis 28 of the inner shaft 2, said exposed electrode surface being connected, by way of an adapter 32, to the distal end 31 of the second electrode 5 on the inner side 30 of the insulating insert.

According to the exemplary embodiments, the first electrode 4 is designed as an active cutting electrode while the exposed electrode surface 29 of the insulating insert 8 is designed as a passive neutral electrode to permit return flow of the high-frequency current.

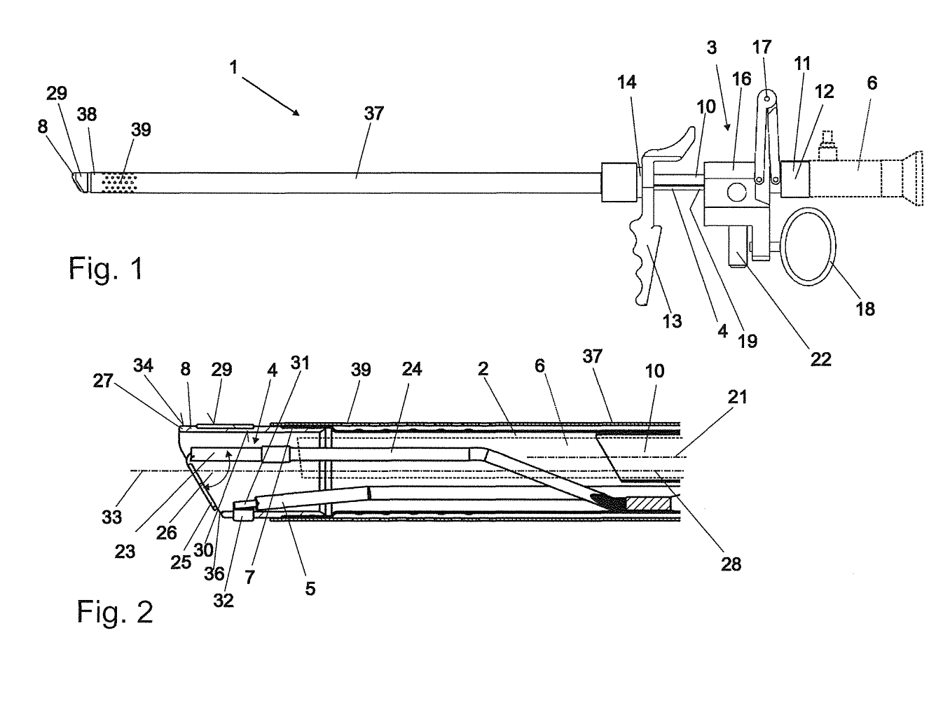

According to the exemplary embodiments in FIGS. 1 to 4, the exposed electrode surface 29 of the insulating insert 8 is arranged on the outer side 34, facing radially away from the longitudinal axis 33, of the insulating insert 8, with the exposed electrode surface 29 of the insulating insert 8 being electrically insulated toward the inside, i.e. toward the inner side 30 of the insulating insert 8.

According to the exemplary embodiments in FIGS. 1 and 2, the electrode surface 29 is wider on the roof-shaped upper side of the insulating insert 8 and narrower in the area of the underside, i.e. in the area of the cutting edge 36.

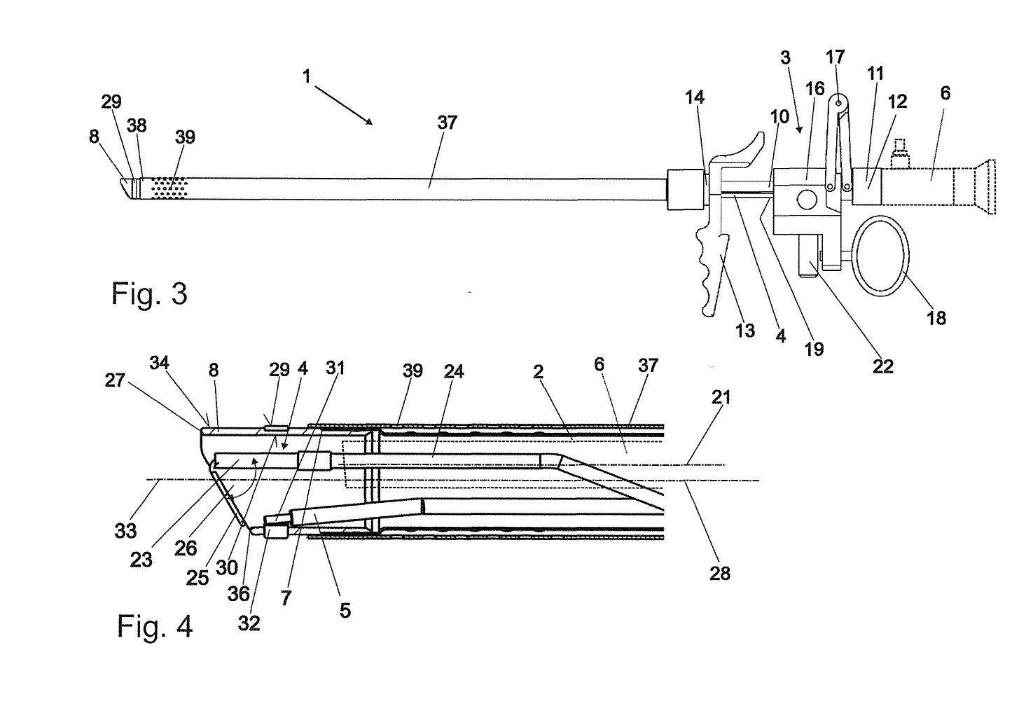

According to the exemplary embodiments in FIGS. 3 and 4, the exposed electrode surface 29 of the insulating insert 8 is ring-shaped, i.e. the electrode surface 29 is designed to be of equal width both in the area of the roof as well as in the area of the cutting edge 36.

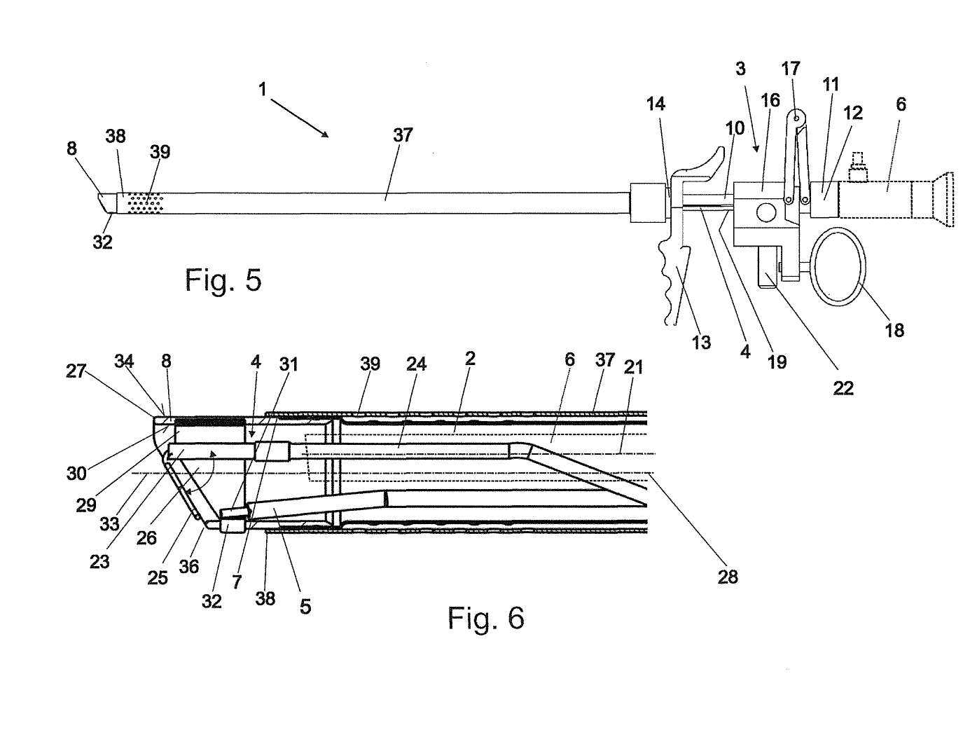

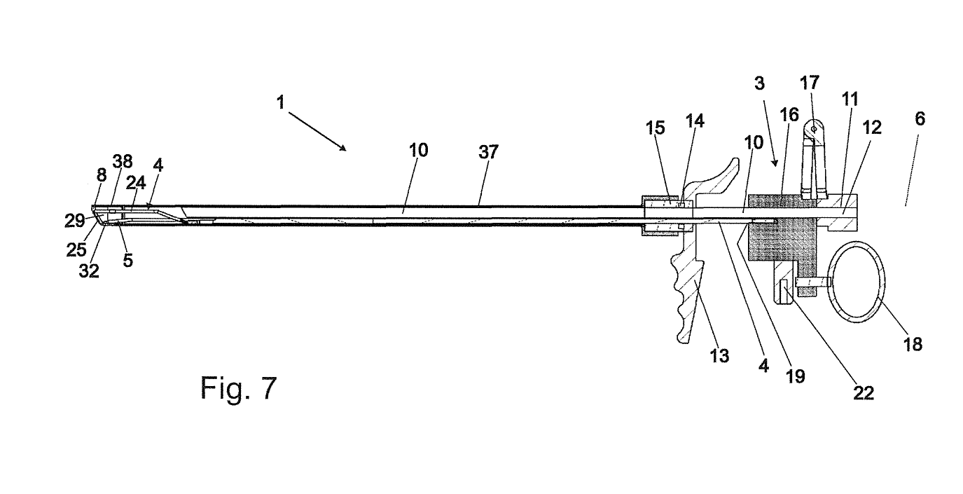

According to the exemplary embodiments in FIGS. 5 to 7, the exposed electrode surface 29 of the insulating insert 8 is arranged on the inner side 30 of the insulating insert 8, facing the longitudinal axis 33 in a radial direction, and the exposed electrode surface 29 of the insulating insert 8 is electrically insulated toward the outside in a radial direction.

According to the exemplary embodiments, the exposed electrode surface 29 is arranged in an insulated manner in a longitudinal direction, on the one hand toward the distal end 27 of the insulating insert 8, and on the other hand toward the free or distal end 7 of the inner shaft.

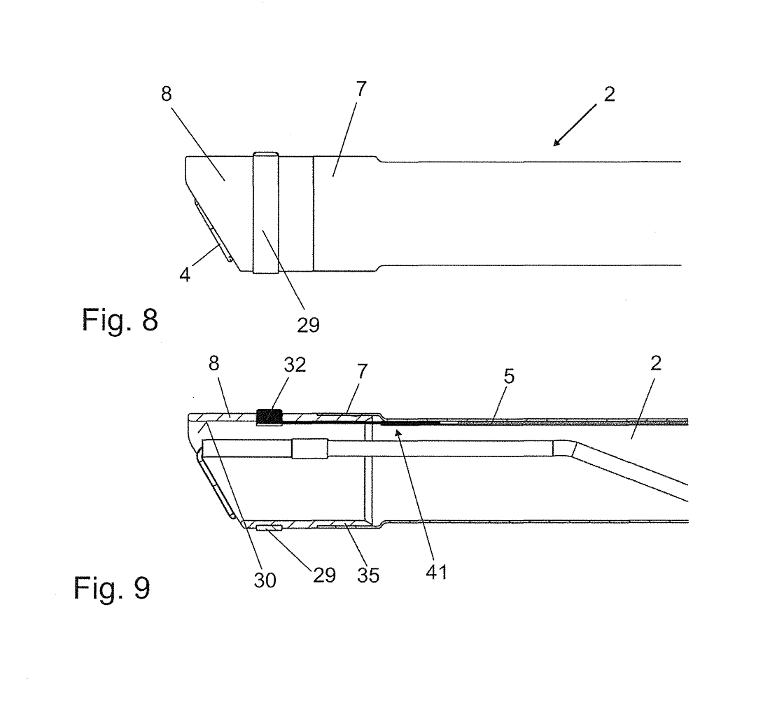

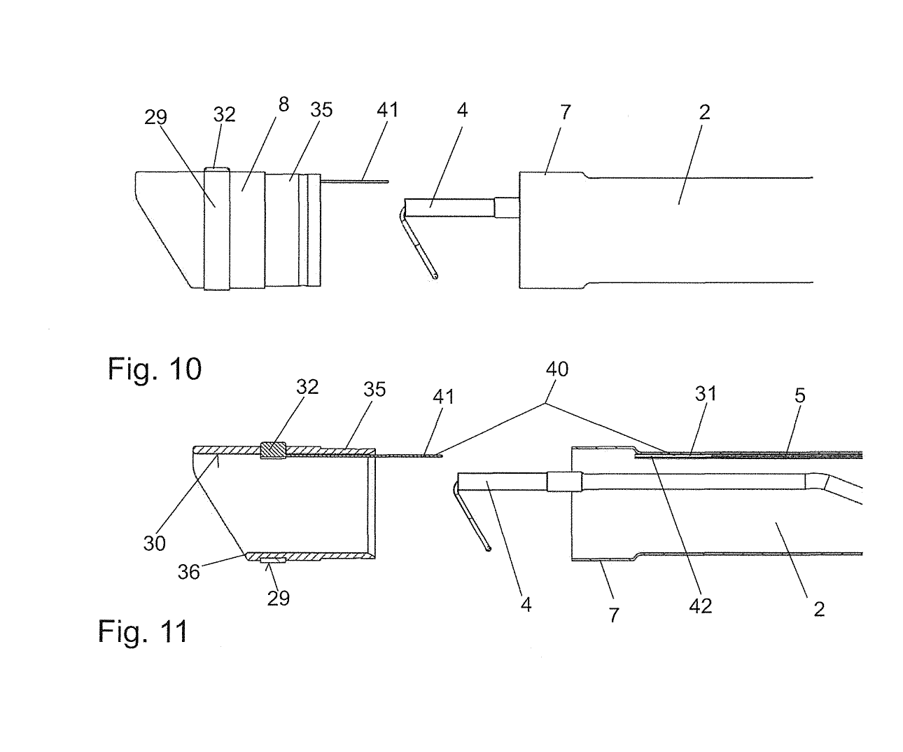

According to the exemplary embodiments in FIGS. 8 to 11, the proximal end 35 of the insulating insert 8 is inserted into the distal end 7 of the inner shaft 2. The adapter 32 connected to the electrode surface 29 is connected to the distal end 31 of the second electrode 5 by way of a plug connector 40. The plug connector 40 consists of a plug contact 41, which protrudes from the proximal end 35 of the insulating insert 8 and is connected via an electrically conductive connection to the adapter 32, and also of a contact sleeve 42, which forms the distal end 31 of the second electrode 5, and which accommodates the free end of the plug contact 41.

The insulating insert 8 can be made of plastic, for example, with the exposed electrode surface 29 being made of a metallic material. The insulating insert 8 can however also be made of a non-conductive ceramic material, with the exposed electrode surface 29 being made of a metalized ceramic material.

According to the exemplary embodiments, the inner shaft 2 is arranged within an outer shaft 37, with the two shafts 2, 37 forming a continuous flushing shaft with continuous flushing. In this connection, the distal end 38 of the outer shaft 37 is set back, i.e. spaced at a distance, relative to the distal end 27 of the insulating insert 8 in the proximal direction, in the area of the insulating insert 8.

The distal end 38 of the outer shaft 37 has a plurality of return flow openings 39.

According to the exemplary embodiments, both the inner shaft 2 and the outer shaft 37 are made of an electrically conductive material.

Of course, the embodiments discussed in the specific description and shown in the Figures are merely illustrative exemplary embodiments of the present invention. In the light of the present disclosure a person skilled in the art has a broad spectrum of optional variations available. In particular, the passive electrode transporter shown in the exemplary embodiment can also be designed as an active electrode transporter known to a person skilled in the art. Of course, in order to coagulate tissue, a person skilled in the art can replace the cutting electrode 4 by a coagulation electrode known to a person skilled in the art. It is not necessary for the second electrode 5, which is arranged within the inner shaft 2, to be arranged below, in the area of the cutting edge 36, or above, as shown in the exemplary embodiments. A person skilled in the art will arrange it at a suitable location along the inner shaft 2 in consideration of the arrangement of the optical system and of the electrode guide tube (not shown) of the electrode transporter 3.

### LIST OF REFERENCE NUMBERS

1 bipolar resectoscope
2 inner shaft
3 electrode transporter
4 first electrode
5 second electrode
6 optical system
7 distal end of 2
8 insulating insert of 2
9 proximal end of 2
10 guide tube of 3
11 proximal end of 3
12 connector piece of 3
13 finger grip of 3
14 connector body of 3
15 main component of 2
16 sliding body of 3
17 joint of 3
18 thumb ring of 16
19 proximal end of 4
20 guide channel of 16
21 longitudinal axis of 10
22 plug socket
23 distal end of 4
24 loop guide tube of 4
25 cutting loop
26 angle of 25
27 distal end of 8
28 longitudinal axis of 2
29 electrode surface of 8
30 inner side of 8
31 distal end of 5
32 adapter of 5 and 8
33 longitudinal axis of 8
34 outer side of 8
35 proximal end of 8
36 cutting edge of 8
37 outer shaft
38 distal end of 37
39 return flow openings
40 plug connection
41 plug contact of 8
42 contact sleeve of 4

1. A bipolar resectoscope (1), comprising
an inner shaft (2) having at its distal end (7) an insulating insert (8) made of an electrically non-conductive material,
an electrode transporter (3) that can be arranged in the inner shaft (2),
a first electrode (4), which can be arranged in the electrode transporter (3) in such a way that it is longitudinally movable, and which can be connected at its proximal end (19), facing away from the distal end, to a first connection of a high-frequency generator, and which is bifurcated at its distal end (23) and has, parallel to the longitudinal axis (21), two parallel loop guide tubes (24), between which a semicircular cutting loop (25) is tensioned, and
a second electrode (5), which can be connected at its proximal end to a second connection of the high-frequency generator
characterized in that
at its distal end (27) the insulating insert (8) has a circumferential electrically conductive electrode surface (29) that is exposed transversely to the longitudinal axis (28) of the inner shaft (2), the exposed electrode surface being connected to the distal end (31) of the second electrode (5) on the inner side (30) of the insulating insert (8), and in that the exposed electrode surface (29) of the insulating insert (8) is arranged, in the longitudinal direction, on the one hand toward the distal end (27) of the insulating insert (8), and on the other hand toward the free end (7) of the inner shaft (2), and in that the exposed electrode surface (29) of the insulating insert (8) is arranged on the outer side (34), facing radially away from the longitudinal axis (33), of the insulating insert (8), and is electrically insulated toward the inside, or in that the exposed electrode surface (29) of the insulating insert (8) is arranged on the inner side (30), facing radially towards the longitudinal axis (33), of the insulating insert (8), and is electrically insulated toward the outside.

2. The bipolar resectoscope of claim 1, wherein
the first electrode (4) is designed as an active cutting electrode and the exposed electrode surface (29) of the insulating insert (8) is designed as a passive neutral electrode.

3. (canceled)

4. The bipolar resectoscope of claim 1, wherein
the insulating insert (8) is made of plastic and that the exposed electrode surface (29) is made of a metallic material.

5. The bipolar resectoscope of claim 1, wherein
the insulating insert (8) is made of a ceramic material and
the exposed electrode surface (29) is made of a metalized ceramic material.

6. The bipolar resectoscope of claim 1, wherein
the inner shaft (2) can be arranged within an outer shaft (37) and the inner and outer shafts (2, 37) form a continuous flushing shaft with continuous flushing.

7. The bipolar resectoscope of claim 6, wherein
the distal end (38) of the outer shaft (37) is set back relative to the distal end (27) of the insulating insert (8) in the proximal direction, in the area of the insulating insert (8).

8. The bipolar resectoscope of claim 6, wherein
the distal end (38) of the outer shaft (37) has a plurality of return flow openings (39).

9. The bipolar resectoscope of claim 1 wherein
the inner shaft (2) is made of an electrically conductive material.

10. The bipolar resectoscope of claim 1, wherein
the proximal-side end (35) of the insulating insert (8) is inserted into the distal end (7) of the inner shaft (2) and in that
the adapter (32) connected to the electrode surface (29) is connected to the distal end (31) of the second electrode (5) by means of a plug connector (40).
